(19) United States
(12) Patent Application Publication
(10) Pub. No.: US 2008/0086110 A1
(43) Pub. Date: Apr. 10, 2008

(54) Extendable Device On An Aspiration Catheter

(76) Inventors: Jason A. Galdonik, Hanover, MN (US); Peter S. Dardi, Atlanta, GA (US); Matthew F. Ogle, Fitchburg, WI (US); Gregory A. Boldenow, Saint Michael, MN (US); Edward J. Anderson, Maple Grove, MN (US)

Correspondence Address: DARDI & ASSOCIATES, PLLC, 220 S. 6TH ST., SUITE 2000, U.S. BANK PLAZA, MINNEAPOLIS, MN, 55402, US

(21) Appl. No.: 11/791,071

(22) PCT Filed: Nov. 21, 2005
(86) PCT No.: PCT/US05/42509

§ 371 Date: Jun. 29, 2007

Related U.S. Application Data
(60) Provisional application No. 60/629,394, filed on Nov. 19, 2004.

(51) Int. Cl. A61M 31/00; A61F 2/06; A61M 25/00

(52) U.S. Cl. 604/509; 604/131; 604/510; 604/513; 623/1.15

(57) ABSTRACT

Aspiration catheters generally comprise a tube or shaft with an aspiration opening at or near its distal end and a suction device that is at or near the proximal end of the catheter. An aspiration lumen extends from the suction device to the aspiration opening. The aspiration catheter can be associated with an extendable device such that their functions can be combined advantageously. The extendable device can function as a treatment structure and/or as a flow modifier. A treatment structure can be used to expand a constriction in a vessel or to deliver a treatment element into the vessel. A flow modifier generally controls the flow to decrease flow in the vicinity of the aspiration opening to improve the efficiency of the aspiration. Various relationships between the extendable structure and the aspiration catheter are described. Balloons can be used effectively as treatment structures or flow modifiers, although self-extending stents can be used as treatment structures and flaps or the like can be used as flow modifiers.

### CROSS REFERENCE TO RELATED APPLICATIONS

This application claims priority to copending U.S. Provisional Patent Application Ser. No. 60/629,394 filed on Nov. 19, 2004 to Galdonik et al., entitled “Balloon on an Aspiration Catheter,” incorporated herein by reference.

### FIELD OF THE INVENTION

The invention relates to intervention medical devices for performing less invasive procedures within a patient. More specifically, the invention relates to catheters that combine treatment functions and/or flow modification with aspiration functions to remove debris from a vessel within the patient.

### BACKGROUND OF THE INVENTION

Restrictions of flow within a vascular vessel of a patient can result in heart attacks, stokes, other detrimental consequences, and even death. Blockages in other types of vessels can also cause significant detrimental health effects. To treat these blockages, techniques have been developed to physically open the blockage. These techniques are generally based on an expanding balloon. The balloon can be used in conjunction with delivery of a stent. The market for drug-coated stents is presently growing at a rapid rate. The drug coating is intended to prevent restenosis, i.e., a redevelopment of the blockage at the same site.

However, the treatment process itself can result in complications. A significant reason for ischemic injury during percutaneous procedures can be generation of emboli which block smaller distal vessels. An embolus can be any particle comprising a foreign and/or native material, which enters the vascular system or other vessel of the body with potential to cause occlusion of blood flow. Emboli can be formed from aggregates of fibrin, blood cells or fragments thereof, collagen, cholesterol, plaque, fat, calcified plaque, bubbles, arterial tissue, and/or other miscellaneous fragments or combinations thereof. Emboli can lodge in the narrowing regions of medium size blood vessels that feed the major organs. Loss of blood flow to surrounding tissue causes localized cell death or microinfarcts. Cerebral microinfarcts can cause stroke leading to confusion, disturbance of speech, paralysis, visual disturbances, balance disturbances and even death. In the heart, emboli can cause myocardial infarcts, i.e. heart attacks. Myocardial infarction refers to the death of a section of myocardium or middle layer of the heart muscle. Myocardial infarction can result from at least partial blockage of the coronary artery or its branches. Blockage of capillaries associated with the coronary arteries can result in corresponding microinfarctions/microinfarcts. Resulting impairments are frequently short term but can be permanent.

One approach to curb complications from emboli formation in procedures has been to use pharmacological therapies during the time of the intervention. Limited therapeutic success has been reported with the use of calcium channel blockers, adenosine, and sodium nitroprusside (Webb, J G, Carere, R G, Virmani, R, Baim, D, Teirstein, P S, Whitlow, P, McQueen, C, Kolodgie, F D, Buller, E, Dodek, A, Mancini, G B, & Oesterle, S: Retrieval and analysis of particulate debris after saphenous vein graft intervention. J Am Coll Cardiol 2000, 34:468-475, incorporation herein by reference.). Glyoprotein IIb/IIIa inhibitors have been used for percutaneous coronary interventions to reduce platelet aggregation, but also fail to show meaningful long term clinical benefit. (Mathew, V, Grill, D E, Scott, C G, Grantham, J A, Ting, H H, Garratt, K N, & Holmes, D R, Jr. The influence of abciximab use on clinical outcome after aortocoronary vein graft interventions. J Am Coll Cardiol 1999, 34:1163-1169 and Mak, K H, Challapalli, R, Eisenberg, M J, Anderson, K M, Califf, R M, & Topol, E J: Effect of platelet glycoprotein IIb/IIIa receptor inhibition on distal embolization during percutaneous revascularization of aortocoronary saphenous vein grafts. EPIC Investigators. Evaluation of IIb/IIIa platelet receptor antagonist 7E3 in Preventing Ischemic Complications. Am J Cardiol 1997, 80:985-988, both of which are incorporated herein by reference.) Since embolization often develops from physical disruption of fibrotic plaque, a mechanism of therapeutic embolic protection specifically targeted at prevention of platelet aggregation and blood clotting may have little effect on these already-formed, embolizable plaques.

Surgical procedures for the treatment of renal artery stenosis can also generate emboli. There is clinical evidence to suggest that 36% of those treated suffer arterioloar nephrosclerosis caused by atheroemboli. Five-year survival of patients with atheroembolic events is significantly worse than of patients without atheroemboli (54% vs. 85% respectively) [Krishmamurthi et al. J Urol. 1999, 161:1093-6]. These patients could also benefit from distal protection devices. Foreign material in the stream of flow can cause turbulence or low flow. Such flow conditions have been shown to increase rates of infection. Thrombus not only generates emboli, but also increases the risk of infection.

### SUMMARY OF THE INVENTION

In a first aspect, the invention pertains to a method for retrieving an embolism protection device having a low profile configuration and an extended deployed configuration. The method comprises withdrawing the embolism protection device into an aspiration catheter within a patient's vessel while aspiration is being applied. An extendable device associated with the aspiration catheter is extended and partially occludes flow relative to the flow in which the extendable device does not have an extended configuration.

In another aspect, the invention pertains to an aspiration catheter comprising a suction connection, a proximal portion operably connected to the suction connection, a shaft and a self-extending structure. The shaft is operably connected to the proximal portion and has an aspiration opening connected to an aspiration lumen in fluid communication with the suction connection. Generally, in appropriate embodiments, the self-extending structure is within about 20 centimeters of the aspiration opening.

Moreover, the invention pertains to a medical device for performing less invasive medical procedures within a patient comprising an aspiration catheter and a balloon apparatus comprising a balloon element and a balloon catheter. The balloon catheter is disconnected from the aspiration catheter. Also, the balloon element is mounted on the aspiration catheter.

In a further aspect, the invention pertains to a medical device for performing less invasive medical procedures within a patient, the device comprising an aspiration catheter, a balloon apparatus comprising a balloon element, and a balloon catheter. The aspiration catheter has an aspiration lumen operably connected to a suction device and an aspiration opening at the distal end of the aspiration lumen. Furthermore, the aspiration opening is distal to the balloon element, and the aspiration opening has an area greater than the average cross section of the aspiration lumen.

In another aspect, the invention pertains to a medical device for performing less invasive medical procedures within a patient, the device comprising a guide structure, an aspiration catheter, a balloon element mounted on the aspiration catheter and a fluid source operably connected to the balloon element. The aspiration catheter comprises a suction device, a proximal portion, a rapid exchange segment having a distal end and a shaft attached between the proximal portion and the rapid exchange segment. The rapid exchange segment comprises a port adjacent the connection between the shaft and the rapid exchange segment. In addition, the guide structure passes through the port, and the rapid exchange segment has a single lumen.

In addition, the invention pertains to a medical device for performing less invasive medical procedures within a patient, the device comprising a guide structure, a filter element connected to the guide structure, an aspiration catheter and an extendable element mounted on the aspiration catheter. The aspiration catheter comprises a suction connection and a distal opening into an aspiration lumen in fluid communication with the suction connection. The aspiration catheter is configured to ride over the guide structure. The filter element has a low profile configuration and a deployed configuration extending outward from the guide structure. The filter in the low profile configuration substantially fits into the distal opening.

Furthermore, the invention pertains to a medical device comprising an aspiration catheter, a treatment catheter and a guide structure. The aspiration catheter comprises a rapid exchange segment, a proximal section, a shaft connected between the rapid exchange segment and the proximal section to form an aspiration lumen extending between the proximal section and the rapid exchange section, and a suction device connected to the proximal section and in fluid communication with the aspiration lumen. The rapid exchange segment comprises a treatment catheter port, and the treatment catheter comprising a shaft, a treatment structure mounted on the shaft and an actuation device operably connected to the treatment structure. The shaft of the treatment catheter extends through the treatment catheter port.

In an addition aspect, the invention pertains to a method for treating a flow restriction in a vessel of a patient. The method comprises positioning an aspiration catheter past the restriction along the flow and inflating an expansive element at the restriction to expand the vessel at the restriction while applying aspiration through the aspiration catheter. Generally, the flow through the vessel is unobstructed by artificial mean prior to inflation of the expansive element and immediately following deflation of the expansive element.

Moreover, the invention pertains to a method for forming a medical device for placement in a vessel of a patient. The method comprises reversibly attaching a balloon element on the exterior of an aspiration catheter.

### BRIEF DESCRIPTION OF THE DRAWINGS

FIG. 1 is a schematic side view of combined medical device comprising an aspiration catheter and a balloon with phantom lines showing hidden structure.

FIG. 2 is a fragmentary view of the distal end of the device of FIG. 1 in which the aspiration catheter has a distal compartment to assist with the removal of an embolism protection device.

FIG. 3A is a fragmentary view of the distal end of the device of FIG. 1 depicting the balloon in an expanded configuration.

FIG. 3B is a fragmentary view of the distal end of an alternative embodiment of the device of FIG. 1 with a balloon that does not extend around the entire circumference of the catheter.

FIG. 4 is a sectional view of the aspiration catheter of FIG. 1 depicting one embodiment of the cross section, the section being taken along line a-a of FIG. 1.

FIG. 5 is a sectional view of an alternative embodiment of the cross section of the aspiration catheter of FIG. 1, the section being taken along the line a-a of FIG. 1.

FIG. 6 is a fragmentary view of the device of FIG. 1 depicting one embodiment of a connection between the balloon and the balloon lumen with hidden structure depicted with phantom lines.

FIG. 7 is a view of a balloon connected to a tube for incorporation into a combined medical device.

FIG. 8 is a sectional side view of a specific embodiment of an integrated guiding device.

FIG. 9 is a schematic, side view of an alternative embodiment of a combined medical device with a balloon catheter separate or disconnected from the aspiration catheter.

FIG. 10 is a sectional view of the device of FIG. 9 with the section taken along line b-b of FIG. 9.

FIG. 11 is a sectional view of the device of FIG. 9 with an alternative sectional structure, in which the section taken along line b-b of FIG. 9.

FIG. 12 is a fragmentary, schematic side view of a balloon structure that can be placed over an aspiration catheter.

FIG. 13 is a schematic, side view of another alternative embodiment of a combined medical device in which an aspiration catheter has a rapid exchange configuration with respect to a guiding structure.

FIG. 14 is a sectional view of one cross section embodiment of the aspiration catheter of FIG. 13, with the cross section taken along line c-c of FIG. 13.

FIG. 15 is a sectional view of an alternative cross section embodiment of the aspiration catheter of FIG. 13, with the cross section taken along line c-c of FIG. 13.

FIG. 16 is a sectional view of a second alternative cross section embodiment of the aspiration catheter of FIG. 13, with the cross section taken along line c-c of FIG. 13.

FIG. 17 is a sectional view of a third alternative cross section embodiment of the aspiration catheter of FIG. 13, with the cross section taken along line c-c of FIG. 13.

FIG. 18 is a fragmentary, side view of the distal end of a combined medical device with three extendable structures.

FIG. 19 is a sectional view of a catheter associated with the device of FIG. 18, the section being taken along line 19-19 of FIG. 18.

FIG. 20 is a schematic side view of a combined medical device with a self-extending stent and an aspiration catheter.

FIG. 21 is a fragmentary, schematic side view of an alternative embodiment of a combined medical device with a self-extending stent.

FIG. 22 is a fragmentary view of the distal end of an embodiment of an aspiration catheter with a self-extending flow modifier with an optional sheath.

FIG. 23 is a fragmentary view of the distal end of an alternative embodiment of an aspiration catheter with a self-extending flow modifier in a low profile configuration within a sheath.

FIG. 24 is a fragmentary view of the distal end of the aspiration catheter of FIG. 23 with the flow modifier in an extended deployed configuration.

FIG. 25 is a fragmentary view of the distal end of an embodiment of an aspiration catheter with a self-extending, flaring flow modifier, in which a sheath is restraining the flow modifier at its proximal side.

FIG. 26 is a fragmentary view of the aspiration catheter of FIG. 25 in which the flow modifier is flared into its deployed configuration with the withdrawal of the sheath.

FIG. 27 is a fragmentary view of the distal end of an aspiration catheter with a self-extending flow modifier that extends only partially around the circumference of the catheter.

FIG. 28 is a fragmentary view of the distal end of an aspiration catheter having a deformable flow modifier and a sheath that engages the flow modifier to deploy the flow modifier.

FIG. 29 is a fragmentary view of the distal end of an aspiration catheter with a flow modifier attached to a pull wire that can control the configuration of the flow modifier, in which the flow modifier is shown in a low profile configuration.

FIG. 30 is a fragmentary view of the distal end of the aspiration catheter of FIG. 29 in which the flow modifier is in an extended, deployed configuration.

FIG. 31A is a fragmentary view of the distal end of an aspiration catheter with a helical flow modifier.

FIG. 31B is a sectional view of the aspiration catheter of claim 31A taken along line B-B of FIG. 31A.

FIG. 32A is a fragmentary view of the distal end of an aspiration catheter with three helical flow modifiers distributed around the circumference of the catheter.

FIG. 32B is a sectional view of the aspiration catheter of FIG. 32A taken along line B-B of FIG. 32A.

FIG. 33 is a fragmentary view of the distal end of an aspiration catheter with a flow modifier that is engageable with the rotation of a sheath.

FIG. 34 is a fragmentary view of the distal end of an aspiration catheter with an expandable balloon that extends only partially around the circumference of the catheter, in which the balloon is depicted in an un-inflated configuration.

FIG. 35 is a fragmentary view of the aspiration catheter of FIG. 34 in which the balloon in inflated.

FIG. 36 is a side perspective view of an aspiration catheter configured to be associated in a rapid exchange configuration with a balloon catheter, which rides over a guide structure.

FIG. 37 is a side perspective view of an aspiration catheter configured to be associated in a rapid exchange configuration with a balloon catheter, which is configured in a rapid exchange configuration with respect to a guide structure.

### DETAILED DESCRIPTION OF THE INVENTION

Improved combined medical devices described herein provide for the delivery of an extendable element in association with an aspiration catheter. In some embodiments of the combined medical device, the aspiration lumen is positioned after delivery to aspirate emboli generated by deployment of the extendable element, such as a balloon or a self-extending stent, which is intended to make contact with the vessel walls to open up a blockage in the vessel. The extendable elements that function to open blockages in a vessel are referred to herein as extendable treatment structures. The various embodiments of the medical devices with extendable treatment structures provide for easier delivery and use of the products that can shorten the time of the procedure and correspondingly reduce costs while improving outcomes. In alternative embodiments, rather than having a treatment structure mounted on an aspiration catheter, a treatment catheter with a treatment structure extends through a rapid exchange port on the aspiration catheter. In additional or alternative embodiments, an extendable element partially occludes, i.e., blocks, flow within the vessel rather than necessarily contacting the vessel walls. The partially occlusive extendable devices can improve aspiration function and are referred to here as extendable flow modifiers. Extendable elements herein without further qualification refer generally to both extendable treatment structures and extendable flow modifiers. In some embodiments, the extendable elements may be reversably mounted piggyback on the aspiration catheter.

Suitable vessels for the delivery of the combined devices include, for example, blood vessels, such as arteries or veins, bile ducts, urinary track vessels, or any other suitable vessel in a patient. Suitable patients include, for example, mammals, such as domestic pets, farm animals, and in particular humans. The devices described herein can be used in percutaneous procedures in which the devices are inserted into orifices or through small incisions or in surgical procedures in which a vessel is exposed in the procedure.

In some embodiments, a treatment structure, such as a balloon, and an aspiration catheter are independently connected to separate lumens that are disconnected from each other while providing for their separate functions. Similarly, in some embodiments, separate lumens and/or separate actuation elements can be used to actuate the extendable flow modifiers. Also, in some embodiments, the aspiration catheter has a rapid exchange segment with a suitable port such that the aspiration catheter can ride on a guide structure in a rapid exchange configuration. The combined medical devices optionally can also have a filter element connected to a guide structure over which the aspiration catheter with an associated balloon, other treatment structure or other extendable flow modifier can be delivered, although an occlusive embolism protection structure can be used in some embodiments. Generally, the aspiration catheters can be effectively used in the retrieval of the filter device. Corresponding improved medical procedures performed with these combined devices are described. These combined devices are extremely versatile in the sense that they can be easier to use than corresponding separate structures and can provide consistent results based on an inherently improved configuration within the combined device. In some embodiments, the medical devices are used in conjunction with separate treatment structures, which can be balloons, stents or the like.

In some embodiments, the types of devices and procedures described herein relate to combined structures with both treatment and prevention instruments that can be simultaneously deployed and used very efficiently. In particular, a treatment device with an extendable element, such as a balloon, can be coupled to an aspiration catheter, generally with the extendable element riding piggyback on the aspiration catheter. A treatment device generally extends to open up correspondingly the vessel at a restricted flow area. For example, the treatment device can be a balloon that is inflated to expand a restricted region of a vessel. Inflation of the balloon can be coupled with the delivery of a stent in conjunction with expansion of the balloon. Similarly, expansion of the balloon can be coupled with delivery of a therapeutic agent during delivery of the balloon. In other embodiments, the extendable structure can be, for example, a self-extending stent. Restricted flow regions can be the result, for example, of plaque deposition on the wall of the vessel, deposition of debris that was generated down stream form the blockage site, an injury to the vessel or any other event that results in vessel blockage.

In treatment structure-based embodiments, the combined medical devices described herein have an aspiration catheter with a treatment structure with an extendable element, such as a balloon, mounted on the aspiration catheter. Several different configurations can provide improved procedures for use of the devices. In appropriate embodiments, the aspiration catheter and a balloon can be in communication with separate lumen that each extends roughly between a location near the proximal end of the combined device and the particular component at or near the distal end of the device. In some representative embodiments, the aspiration catheter has an aspiration opening distal relative to a balloon, such as at the distal tip of the catheter. By locating the opening of the aspiration catheter downstream from the balloon, the natural flow in the vessel tends to move any debris toward the catheter opening such that it can be removed with less volume of liquid. In some embodiments, the combined device has a catheter with a central lumen operably connected to a suction device, a balloon mounted on the catheter and a balloon lumen positioned radially outward relative to the central lumen.

In embodiments relating to a flow modifier, the extendable device is selected to reduce the flow through the vessel without stopping the flow. The flow through a blood vessel is pulsatile corresponding with the heart pulses. The flow rate then has a maximum and minimum value over the course of a pulse cycle. In general, it is desirable for the aspiration rate to be at least as high as the average low flow values over the cycles to reduce the chance of emboli not being captured by the aspiration. Of course, the particular flow rates depend significantly on the particular vessel, and the size of the vessel is only one factor of many in determining the flow rate. However, larger vessels tend to have larger flows. The aspiration rate can be increased by increasing the size of the catheter lumen. A larger catheter lumen can increase the volume of fluid that can be evacuated for a particular period of time. However, arbitrarily large aspiration catheters can be impractical since the catheters are used with other conventional devices, such as guide catheters, hemostatic valves and the like. Also, larger catheters can be disadvantageous due to their stiffness, and the required size of the puncture site.

Therefore, for aspiration in certain vessels, it is desirable to reduce the natural flow in the vessel to improve the aspiration efficiency. The reduction in natural flow can be accomplished through selected impediment of flow within the vessel using a flow modifier. In other words, a flow modifier slows the flow rate through the vessel, such as through reducing the effective flow lumen with a partially occluding volume or flow diversion. For given pressures in the vessel, an effective reduction in the vessel diameter or vessel pressure results in a corresponding reduction in flow. Suitable extendable devices that function as flow modifiers include, for example, smaller volume balloons with either extension around the catheter's entire circumference or only a portion of the catheter's circumference, flaps, self-extending structures optionally with membranes, flow diverters, such as spiral fins or the like that effectively induce a pressure drop, or the like. Self-extending structures can be delivered and/or retrieved with a sheath, pull wires, push wires, obturators and the like.

In some embodiments, the aspiration catheter rides on a guide structure with a filter at the distal end of the guide structure. Thus, the filter provides added protection in combination with the aspiration catheter. In some embodiments, the filter can capture the debris when it is generated, and the aspiration catheter can collect and debris that is released during retrieval of the filter. This combined structure is particularly efficient in its delivery and use. The aspiration catheter can have a compartment at its distal end for withdrawing the filter during its removal from the patient. This distal compartment can be adjacent the balloon or the balloon can ride over the distal compartment. Aspiration catheters with improved tracking can comprise a curved tip, a distal guide lumen and/or a bumper that retractably extends from the aspiration port to facilitate tracking of the aspiration catheter along a guide structure. Improved tracking aspiration catheters are described further in copending U.S. patent application Ser. No. 11/207,169 to Boldenow et al., entitled “Improved Tracking Aspiration Catheter,” incorporated herein by reference. These improved tracking features can be combined with any suitable embodiment below, as appropriate and desired.

In additional or alternative embodiments, the aspiration catheter and a balloon are connected to respective lumens that are physically disconnected. The balloon can function as a treatment structure or a flow modifier. By using separate lumens, a simpler structure can be used for the respective lumens without the need for isolated separate lumens within a single catheter. Of course, the separate lumens for the catheter and the balloon can be superficially attached for convenient handling such as with bands. The balloon may be reversibly attached to the aspiration catheter, which is particularly straightforward if the lumens are separate.

The aspiration catheter can provide suction through an aspiration lumen to one or more aspiration openings at or near the distal end of the aspiration catheter. The suction is generally delivered with an appropriate suction device, such as a syringe or pump, that is connected at the proximal end of the aspiration catheter. An aspiration lumen extends through the catheter from at or near the proximal end to at or near the distal end. In addition, the aspiration catheter can have a specific compartment at its distal end for the withdrawal of an embolism protection device such that the compartment functions as a sheath for the embolism protection device as the device is withdrawn. Generally, a suitable compartment can be a distal portion with an enlarged diameter. A similar compartment can be used even if an embolism protection device is not recovered within the compartment since the enlarged diameter compartment can function to reduce flow through the vessel near the aspiration opening. The aspiration catheter can comprise a radiopaque marker to facilitate the positioning of the device within the patient.

The aspiration catheter can be an over the wire design in which the catheter runs over the wire along the entire length of the catheter. In alternative embodiments, the aspiration catheter has a rapid exchange design. In a rapid exchange configuration, the catheter has a rapid exchange segment that rides over a guide structure. In some embodiments, both the distal portion of the aspiration catheter and the balloon can be mounted on the rapid exchange segment such that these elements are properly positioned for use within the vessel. Using a rapid exchange construction provides for a particularly convenient approach for the delivery of both a treatment structure and aspiration catheter over a single guide structure with full rapid exchange convenience. In additional embodiments described further below, the aspiration catheter has a rapid exchange port through which a treatment catheter can pass rather than just a guide structure.

For the rapid exchange designs, a port provides for the transition of the wire out from the catheter. In some embodiments, a channel or open lumen extends from the proximal to the distal ends. For these embodiments, it can be challenging to direct the wire to the port. A grid or the like can be placed over the opening of the lumen near the port to guide the wire to the port without obstructing flow through the catheter during use. Multiple lumens can be constructed such that the wire follows a major lumen out through the side port. Alternatively or additionally, a loading tool can be inserted temporarily through the port to guide the wire to the port. For example, the wire can be directed through the port in a tube or in a cavity at the end of a rod. Once the wire is through the port, the tool can be disconnected from the guide structure.

In embodiments having a balloon, the balloon is generally delivered in a compact configuration for placement at a desired location within a patient's vessel. A fluid is flowed through a lumen to the balloon to expand the balloon. The pressure used to expand the balloon should be sufficient to overcome the vessel pressure, such as blood pressure, while having enough overpressure to perform the desired expansion. For treatment structures comprising a balloon, the expanding balloon correspondingly expand the vessel at a point of restriction. Appropriate pressures for balloon expansion can be evaluated based on well developed skill in the art for particular applications. The material of the balloon should be strong enough not to rupture under the applicable pressures. For relevant embodiments, pressure can be applied repeatedly, if desired by the health care professional to achieve desired vessel opening. When treatment is completed, the balloon is generally deflated for removal from the patient. In some embodiments, the combined medical device can comprise a plurality of balloons, which may or may not be associated with corresponding isolated balloon lumen for inflation of the balloon. A therapeutic agent, such as a restenosis inhibiting agent, can be delivered during the deployment of the balloon, for example, by placing the therapeutic agent on the surface of the balloon. See, for example, U.S. Pat. No. 6,491,617 to Ogle et al., “Medical Articles That Resist Restenosis,” incorporated herein by reference.

Also, the balloon can be a flow modifier that does not apply strong forces to the vessel walls. Balloons that function as flow modifiers can have a smaller volume such that they do not extend to the vessel walls. Additionally or alternatively, flow modifying balloons can have positions on the catheter that do not extend around the entire circumference of the catheter such that the expanded balloon does not extend across the entire vessel lumen. The size shape and positioning of the flow modifying balloon can be selected to induce the desired flow modification.

In addition, the combined device can comprise a balloon for dilation of the vessel along with a stent for delivery into the dilated vessel. The stent may or may not be directly associated with the balloon. For example, the stent can be extended into place during expansion of the balloon. Furthermore, the device can comprise a plurality of balloons, each mounted on the catheter shaft or tube. For example, a first balloon can be used for expanding the vessel while a second balloon can be used to deploy a stent. Similarly, a first balloon can be configured as an extendable treatment structure, while a second balloon can be configured as an extendable flow modifier. Thus, a range of structures can be adapted for use in the combined medical devices for convenient use. The stents can be drug coated as desired, and drug coated stents are commercially available.

In some embodiment with a treatment structure, the treatment structure comprises a self-extending stent without an associated balloon. If the force of the self-extending stent is sufficient, the stent itself opens the occluded vessel during its extension. The self-extending stent can be triggered for deployment, for example, through the withdrawal of a sheath covering the stent and inhibiting the self-extension or through the use of a trigger, for example, that draws the two lateral ends of the stent together to initiate the extension of the stent. The combined medical device can comprise the components used to deploy a self-extending stent. Alternatively or additionally, a self-extending stent can be deployed after a balloon has been used to open the vessel. A self-extending stent can be adjacent the balloon along the piggy back structure.

In some embodiments, a treatment catheter can interface with an aspiration catheter in which the treatment catheter passes through a rapid exchange port in the aspiration catheter rather than mounting the treatment structure on the aspiration catheter. In these embodiments, the treatment structure generally projects outward from the distal end of the aspiration catheter. The rapid exchange port can be slightly larger then the diameter of the shaft of the treatment catheter such that the port is essentially blocked when the treatment catheter extends through the port and aspiration effectively takes place through an aspiration opening, which can be at the distal end of the aspiration catheter. The treatment catheter can ride over a guide structure, or the treatment catheter can has a rapid exchange port such that the treatment catheter can interface with the guide structure in a rapid exchange configuration.

A variety of flow modifier structures can be used in addition to balloon structures. For example, a flow modifier can comprise struts, woven or interlocking structural elements or other structural elements, which may or may not be covered with a membrane to achieve a desired degree of flow modification. In additional or alternative embodiments, the flow modifier comprises a deformable sheet of material that can be transformed between a lower profile delivery configuration and an extending deployed configuration. These mechanical structures can be self-extending, such that once they are released with an appropriate actuation element, they transform to their deployed, extended configuration. A sheath or the like can be used to constrain and release a self-extending element. In other embodiments, a pull-wire, push wire, rod or tube can be used to control the deployment of a flow modifier. The flow modifier may or may not extend around the entire circumference of the aspiration catheter and similarly may or may not have cylindrical symmetry. Several suitable configurations are described below.

A range of designs of the embolism protection device are suitable can be incorporated into the systems described herein. Filtering embolism protection structures with a three dimensional filtering matrix can provide particularly desirable properties. In particular, the filtering matrix can entrap larger emboli on its surface and smaller emboli within the matrix to provide improved filtering with less occlusion of flow. The filter furthermore provides a distribution of effective pore sizes. In addition, a three dimensional filtering matrix generally does not block suction while the device is converted to a recovery configuration. In fiber-based filters described herein, the fiber structures can facilitate fluid motion while trapping small emboli. The three dimensional matrices generally have considerable flexibility to conform to the vessel wall shape to effectively prevent gaps larger than the effective pore sizes of the filter. Also, the three dimensional filters have considerable filtering capacity without becoming blocked due to alternative flow channels through the matrix. Thus, embolism protection devices with a three dimensional filtering matrix can provide improved performance during filtering and/or during removal. Also, it can be desirable to include a radio-opaque marker on the embolism protection device to facilitate positioning of the device.

Certain designs of the embolism protection device can be particularly suitable. For example, in some embodiments, the embolism protection device can be self-extendable upon deployment into a patient's vessel. Such embolism protection devices can comprise polymers that help to effectuate the desired extension/expansion. Specifically, the embolism protection device can comprise a swellable polymer, such as a hydrogel, a shape memory polymer or the like. In some embodiment, the embolism protection device comprises surface capillary fibers. Surface capillary fibers are particularly desirable since they are able to trap smaller emboli within the surface capillaries and larger emboli between the fibers for extremely effective filtering of emboli. The surface capillary fibers can be used in a bundle within the embolism protection device. The number and properties of the fibers can be selected to trap emboli with selected properties while permitting desired flow through the vessel. In embodiments, of interest, the embolism protection device provided for little if any resistance to flow through the vessel.

In some embodiments of interest, the embolism protection device is attached at or near the end of a core wire that is part of an integrated guiding structure. The embolism protection device is then delivered within the vessel with the steerable integrated guiding device. The integrated guiding device can also be used to actuate deployment of the embolism protection device. Suitable integrated guiding devices for actuation of the embolism protection device can comprise a tube over a corewire. The tube can have an outer diameter approximately equal to the outer diameter of a conventional guidewire. To facilitate the conveyance of torque along the corewire, the tube and corewire can be rotationally coupled, at least at selected times. In these embodiments, the corewire can be steered by rotating the tube. The structures can be designed to provide for longitudinal movement of the tube relative to the corewire for actuation of the embolism protection device.

In some embodiments, the embolism protection device comprises a bundle of surface capillary fibers attached at their distal end to a core wire. At their proximal end, the fibers are attached to the tube separated from the corewire by a short tube that rides over the corewire. In these embodiments, moving the tube longitudinally in a distal direction relative to the corewire brings the two ends of the fibers together flaring outward the center of the fibers. Then, the flared fibers can extend across the vessel lumen to filter flow passing through the vessel. To recover the embolism protection device, the fibers can either be distorted into compressed configuration by bending into a sheath to enclose the embolism protection device during recovery, or the device can be extended to a configuration with the fibers more extended. The fibers can be extended for removal by translating the tube in a proximal direction relative to the corewire essentially to un-deploy the embolism protection device. The device with the fibers extended into a lower profile can be drawn into the distal end of an aspiration catheter for removal from the patient in which aspiration during the recovery of the filter into the catheter can reduce or eliminate loss of emboli form the filter. Fiber-based embolic protection devices are described further in copending U.S. patent application Ser. No. 11/072,001 to Galdonik et al., entitled “Steerable Device Having a Corewire Within a Tube and Combination With a Functional Medical Device,” incorporated herein by reference.

Using the combined devices described herein, a plurality of instrumentalities can be simultaneously and conveniently delivered into a patient's vessel. In some embodiments, all necessary components for a selected procedure can be delivered simultaneously. A guide structure, e.g., a guidewire or an integrated guiding structure, generally is first positioned at a desired location within the patient as determined using appropriate imaging techniques. The aspiration catheter and associated extendable element can then be delivered over the guide structure. If a filter is used, it can be deployed before or after the placement of the aspiration catheter.

Aspiration may or may not be initiated prior to deployment of the extendable element. In some embodiments, aspiration is initiated shortly before deployment of a balloon or stent. For embodiments with a balloon as the extendable element, aspiration may be continued for a short period after the balloon deflation or after multiple deployments of the balloon are completed. Aspiration can be stopped once sufficient time has passed after deployment of the balloon that all emboli likely have been aspirated from the vessel. For embodiments in which the extendable element comprises a flow modifier, aspiration is generally initiated after deployment of the flow modifier and stopped before retracting the flow modifier.

In alternative embodiments, an embolism protection device can be deployed prior to deployment of a treatment structure and/or prior to initiating aspiration. For example, an occlusive balloon can be deployed as an embolic protection device to block most or all down stream flow, which is blocked until activation of a treatment balloon or the like is completed. Generally, the occlusive element is deployed until aspiration is completed. In embodiments of particular interest, an embolism protection device comprises a non-occlusive filter that filters flow without blocking, i.e., occluding, flow within the vessel. In filter-based embodiments, aspiration may not be initiated until treatment with a balloon and/or stent is completed and the filter is ready for recovery. The aspiration catheter then functions to capture any emboli released from the filter during recovery of the filter. For appropriate embodiments, aspiration can be stopped once the filter is withdrawn within the distal tip of the aspiration catheter. The selected timing of the application of aspiration can be varied within reasonable ranges. A flow modifier can be used to improve the effectiveness of the aspiration.

To summarize, in embodiments of particular interest, elements can be quickly and conveniently deployed, used and withdrawn using a single integrated system. A filter can be associated with the guiding structure. Using a rapid exchange configuration, the combined aspiration catheter and extendable element can then be quickly delivered over the integrated guiding structure. The filter can be deployed before or after positioning of the aspiration catheter. A treatment structure can be deployed once the filter is deployed and the treatment structure is positioned. Aspiration then facilitated treatment and recovery of the filter with lose of little or no debris as a result of the performing the treatment, and/or aspiration can be used to capture debris as it is generated with the deployment of an extendable treatment structure. A flow modifier can be used to improve the aspiration effectiveness. The entire procedure can be performed quickly, efficiently and more easily than a corresponding procedure without the advantages of the combined structures described herein.

Combined Medical Device Structure

In general, the combined medical device based on an extendable element comprises an aspiration catheter, a suction apparatus, which can be attached at an aspiration connection, an extendable element mounted on the aspiration catheter, and an optional guide structure. The extendable element can be a treatment structure and/or a flow modifier. For embodiments in which the extendable element comprises a balloon, the medical device generally further comprises a balloon lumen and/or a balloon actuator, which can be attached at an actuation connection. The balloon lumen can be within the aspiration catheter or within a distinct balloon catheter. In other embodiments, the extendable element can interface with an actuation tool, such as a sheath, a push rod, a pull rod and the like. In some embodiments, the combined medical device further comprises further medical treatment structures, such as one or more additional balloons, which may be associated with a stent or the like, or a self-extending stent. Similarly, the combined medical device can comprise a flow modifier, which can be an additional balloon, a flap or other appropriate structure, in addition to a treatment structure. In some embodiments, the extendable element can be a self-extending structure, which may or may not be deployed and/or recovered with the assistance of a sheath.

In some embodiments, the combined medical device can further comprise an occlusive structure to function as an embolism protection apparatus. Similarly, the combined medical device can comprise a filter-based (non-occlusive) embolism protection apparatus, which can be associated with the optional guide structure. Desirable filter structures can be based on bundles of fibers, such as surface capillary fibers, in which the deployed filters have little or no pressure drop across the filter.

For appropriate embodiments, various configurations can be used to relate the balloon lumen with the aspiration catheter. In some embodiments, the aspiration catheter can comprise a rapid exchange segment that is further associated with the balloon. Some suitable configurations and some of the associated details of specific embodiments of the structure are described in the following. In further embodiments, the balloon-based treatment structure is replaced with another extendable treatment structure, such as a self-extending stent or a self-extending flow modifying structure. For embodiments with a self-extending stent, the balloon lumen can be replaced with an actuation element, such as a sheath that holds the self-extending stent in an un-deployed configuration until it is withdrawn.

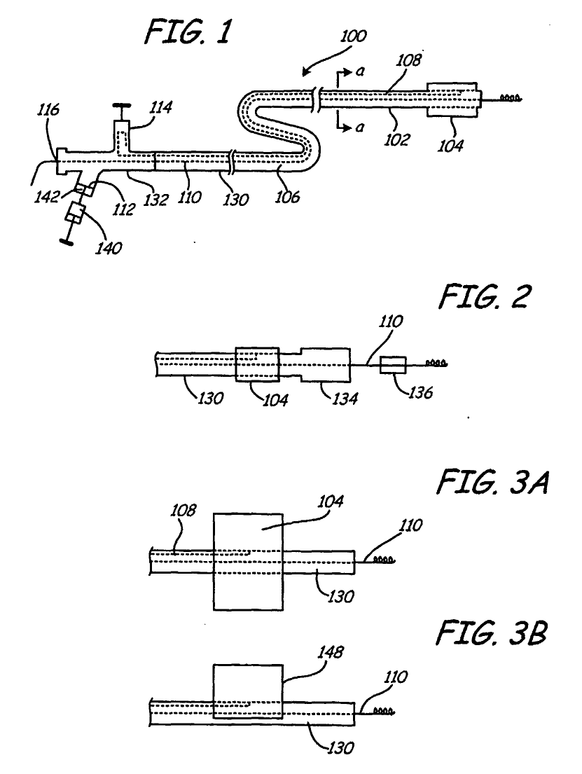

Referring to FIG. 1, an embodiment of a combined medical device 100 with a balloon is shown schematically. Combined medical device 100 comprises aspiration catheter 102, balloon 104, aspiration lumen 106, balloon lumen 108, guide structure 110, aspiration connection 112, balloon actuator 114 and distal port 116. Aspiration catheter 102 comprises a shaft 130 and a proximal section 132. Shaft 130 extends over most of the length of aspiration catheter 102 and comprises the portion of aspiration catheter 102 that extends into the patient. Proximal section 132 generally remains external to the patient and comprises appropriate components for manipulating the aspiration catheter. Proximal section 132 may not be clearly demarcated from the other portions of shaft 130 and may only be identifiable by the respective ports and the like located near the proximal end of the structure.

Shaft 130 can have an approximately constant diameter, a varying diameter and/or sections with different diameters. Shaft 130 is generally relatively flexible for guiding through the vessel with suitable flexible materials described further below, and the flexibility can be selected to be different at different sections of the shaft. In some embodiments, the average outer diameter of shaft 130 ranges from about 0.010 inches to about 0.065 inches and in additional embodiment from about 0.030 inches to about 0.055 inches. For intervention into blood vessels, shaft 130 generally has a length of at least 20 cm, and in some embodiments from about 50 cm to about 300 cm, and in further embodiments from about 100 cm to about 225 cm. A person of ordinary skill in the art will recognize that additional ranges of sizes are contemplated and are within the present disclosure. The aspiration catheter at or near its distal end can comprise a radiopaque marker to provide for visualization using an imaging technique, such as x-ray imaging, for positioning the catheter within the patient.

In some embodiments, as shown in FIG. 2, shaft 130 can have a distal compartment 134 that can facilitate aspiration and/or provide for withdrawal of an embolism protection device 136 for withdrawal from the patient. Distal compartment 134 generally has a larger diameter compared with the adjacent section of shaft 130. In particular, in some embodiments, distal compartment 134 has a diameter from about 200 percent to about 110 percent and in further embodiments from about 175 percent to about 120 percent of the average diameter of the ten centimeters of the shaft adjacent distal compartment 134. Distal compartment 134 can have a length from about 0.2 centimeters (cm) to about 3 cm and in further embodiments from about 0.5 cm to about 2 cm. A person of ordinary skill in the art will recognize that additional ranges of distal compartment dimensions are contemplated and are within the present disclosure.

Proximal end 132 can comprise a handle, ports and/or other convenient control structures for manipulating aspiration catheter 102 and/or for the interface of aspiration catheter 102 with balloon 104 and any other intervention devices. Aspiration connection 112 provides for connection of aspiration catheter 102 with a suction apparatus 140. Aspiration connection 112 can be placed at the proximal end or other location near the proximal end, as convenient. Generally, aspiration connection 112 comprises a fitting 142 or the like to provide a sealed connection with suction device 140. Suitable fittings include, for example, conventional fitting, such as an elastomeric diaphragm through which a syringe needle can be inserted or a Luer lock. Suitable suction devices include, any suction device that can deliver a selected amount of suction, such as a syringe, a compressed bladder, a pump, such as a peristaltic pump or a piston pump, or the like. A tube or the like can be used to connect the suction device to aspiration connection 112.

Balloon 104 can be deployed in a compact configuration. As the name implies, the balloon forms a closed structure around shaft 130 or over a corresponding tube, although balloon 104 can have small holes that leak fluid gradually after the deployment and expansion of the balloon. Balloon has a structure that allows its appropriate expansion. For example, the material of the balloon can be folded in its un-deployed configuration, and/or the balloon can be formed from an elastic material such that the material expands under pressure. The interior of balloon 104 is in fluid communication with balloon lumen 108 such that fluid from balloon lumen 108 can flow into balloon and expand balloon 104 into an expanded configuration, as shown in FIG. 3A.

In some embodiments, the expanded balloon has a roughly cylindrical shape with an outer surface that applies an approximately equivalent force over the cylindrical surface against the occluded portion of the vessel. In additional or alternative embodiments, the balloon can have different shapes that extend around the circumference of the aspiration catheter or around only a portion of the circumference, for example, as shown in FIG. 3B. Referring to FIG. 3B, balloon 148 extends only partially around the circumference of shaft 130.

In general, the dimensions of the balloon can be selected to be appropriate for the particular application. For some embodiments for treatment of occlusions in blood vessels, the lateral extent to the balloon can be less than about 5 centimeters (cm) with a diameter of less than about 0.2 cm and in further embodiments from about 0.1 to about 0.04 cm. A person of ordinary skill in the art will recognize that additional ranges of balloon sizes within the explicit ranges are contemplated and are within the present disclosure. If the vessel is smaller than the balloons fully extended diameter, the vessel constrains the expansion of the balloon with the corresponding pressure applied to the vessel. For flow modification applications, the diameter of the expanded balloon along with the catheter should be somewhat less than the diameter of the vessel, whether or not the balloon extends around the entire circumference. However, suitable dimensions generally depend on the size of the vessels for all applications. Similarly, the extent of the expansion as determined by the volume of fluid delivered to the balloon can be selected to limit the expansion of the balloon.

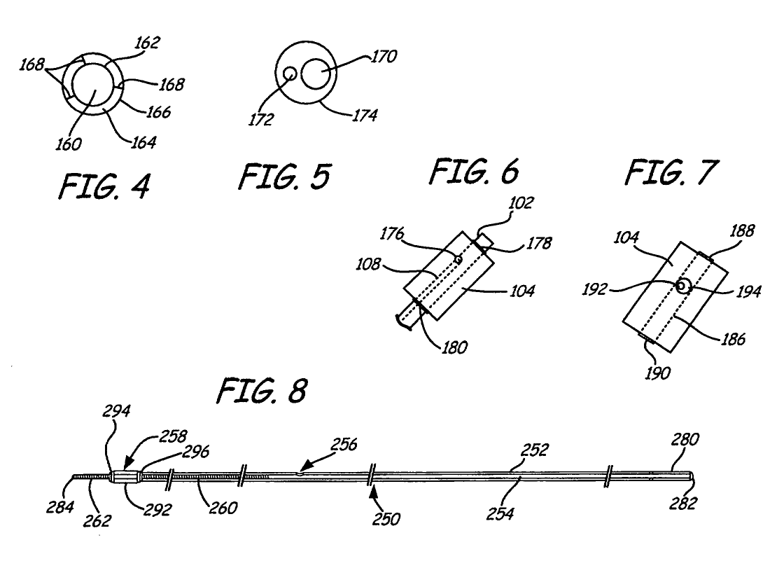

As shown in FIG. 1, balloon lumen 108 is within aspiration catheter 102. Other alternative embodiments are described below. For embodiments in which a balloon lumen is within an aspiration catheter, there are many possible configurations of the balloon lumen relative to the aspiration lumen. Two representative configurations are shown in FIGS. 4 and 5. Referring to FIG. 4, aspiration lumen 160 is a central lumen located within an inner tubular element 162. Balloon lumen 164 is between inner tubular element 162 and outer tubular element 166. Supports 168 can be used to maintain the spatial relationship between inner tubular element 162 and outer tubular element 166. Supports 168 can be positioned periodically or in any other appropriate pattern. Referring to FIG. 5, aspiration lumen 170 and balloon lumen 172 occupy separate channels within aspiration catheter 174. The cross sectional shape and size of aspiration lumen 170 and balloon lumen 172 can be selected appropriately, and the cross sectional shape and size may or may not be constant along the length of aspiration lumen 170.

Referring to FIG. 6, balloon lumen 108 has at least one port 176 connecting balloon lumen 108 with the interior of balloon 104. In some embodiments, a plurality of ports connects balloon lumen 108 with the interior of balloon 104. The plurality of ports may or may not be spaced around the circumference of the aspiration catheter 102. Balloon 104 can be connected with aspiration catheter 102 with seals 178, 180 at the respective ends of balloon 104 if the catheter body forms a portion of the surface of the expanded balloon. For example, seals 178, 180 can be formed from biocompatible adhesives, a heat seal or other appropriate seal.

In alternative embodiments, balloon 104 is sealed to a tube 186 with seals 188, 190, as shown in FIG. 7. Tube 186 has an appropriate diameter to fit over aspiration catheter 102 and has at least one port 192 to establish a flow channel with port 176 leading to balloon lumen 108. An elastic washer 194 or the like can be used to form a fluid tight relationship between the balloon lumen and the interior of the balloon through ports 176 and 192. Brackets, clamps, catches, snaps or the like can be used to reversibly or irreversibly engage tube 186 on aspiration catheter 102 at an appropriate location. For embodiments in which the balloon is releasably mounted on the catheter, the design provides for replacement or selection of the balloon as desired, for example, to select a particular size of balloon.

Guide structure 110 can be a conventional guidewire or a more complex structure. Referring to FIG. 1, a guidewire can comprise a coil 198 at its distal end to facilitate steering of the tip of the guidewire. More complex guide structures can comprise an integrated guide apparatus with a very thin corewire with a tube over the corewire and a torque coupler connecting the corewire and tube. Integrated guiding structures are described further in Published U.S. patent application 2005-0269631A to Galdonik et al., entitled “Steerable Device Having A Corewire Within a Tube and Combination With a Functional Medical Component,” incorporated herein by reference.

In some embodiments, the integrated guiding device comprises a filter system. Referring to a specific representative embodiment in FIG. 8, integrated guiding device 250 comprises tube 252, corewire 254, torque coupler 256, embolism protection device 258, tube coil 260, and wire coil 262. Torque coupler 256 comprises corresponding structural features in tube 252 and corewire 254 that interface to form the torque coupler. This embodiment is dimensioned to reach coronary arteries from a vein in the patient's thigh using conventional catheter procedures.

Corewire 254 can comprise a pull 280 fastened with a solder ball 282 at its proximal end, although other tools can be used to facilitate relative motion of corewire 254 and tube 252. Corewire 254 also comprises a solder ball 284 at its distal end to maintain wire coil 262 on the corewire. Corewire 254 can be coated with a friction reducing agent. In this embodiment, torque coupler 256 limits the longitudinal motion of core wire 254 within tube 252 such that sufficient movement of core wire 254 relative to tube 252 to control embolism protection device 258 while limiting complications due to unwanted movement of corewire 254.

In this embodiment, embolism protection structure 258 comprises a fiber bundle 292 bound with bands 294, 296. One or both of 294, 296 can be formed from a radiopaque material such that it can be viewed using x-rays for determining position within the patient's body, although separate radiopaque bands can be used additionally or alternatively if desired. At its proximal end, the fiber bundle can be bound over a polymer tube that rides over corewire 254. Fiber based embolism protection devices are described further, for example, in copending U.S. patent application Ser. No. 11/072,001 to Galdonik et al., entitled “Steerable Device Having a Corewire Within a Tube and Combination With a Functional Medical Component,” incorporated herein by reference.

Referring to FIG. 1, balloon actuator 114 comprises a fluid that can be directed under pressure through balloon lumen 108. A range of suitable fluids can be used, such as physiologically buffered saline. A biocompatible fluid can be used to avoid health risks if any fluid leaks from the system. Balloon actuator can comprise a syringe, pump or the like, appropriately configured to supply the desired pressure. The fluid reservoir can be reversibly connected to a port with a Leur lock or the like to associate the fluid with the balloon actuator. Proximal port 116 is a suitable port for the passage of guide structure 110 from the catheter without leakage of fluid.

In other embodiments, the balloon lumen is physically distinct from the aspiration catheter. However, the physically distinct balloon lumen can nevertheless touch the aspiration catheter, but the balloon lumen is associated with a balloon catheter that resides exterior to the aspiration catheter. In some embodiments, the balloon catheter can be attached to the exterior of the aspiration catheter, for example, by heat bonding, welding, adhesive bonding, mechanical association with a bracket, a clip, band or the like, or with a combination of these approaches. Using a physically distinct balloon catheter allows for a simpler catheter structure and for the optional attachment of a balloon onto an aspiration catheter such that the aspiration catheter can optionally be used without the balloon.

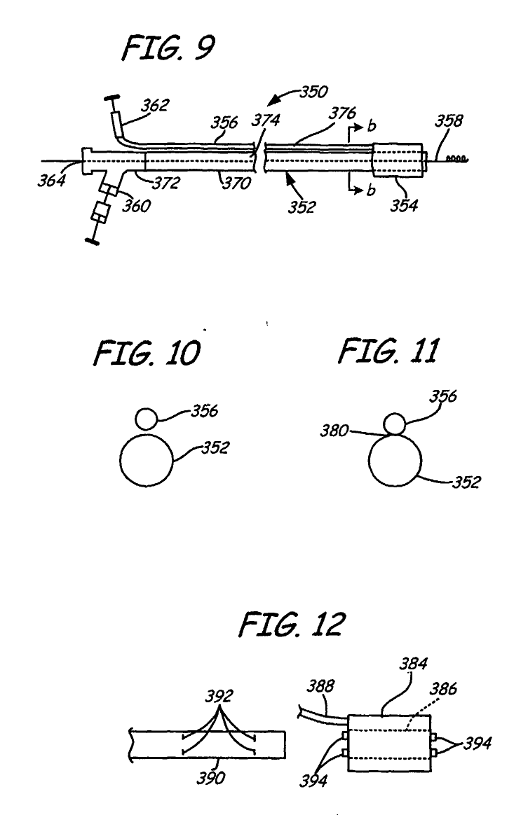

Referring to FIG. 9, combined medical device 350 comprises aspiration catheter 352, balloon 354, balloon catheter 356, guide structure 358, aspiration connection 360, balloon actuator 362 and distal port 364. Aspiration catheter 352 comprises a shaft 370, a proximal section 372 and aspiration lumen 374 that extends within the shaft and proximal section. Balloon catheter 356 has a balloon lumen 376 that connects balloon 354 with balloon actuator 362. Balloon actuator 362 can be a similar structure as balloon actuator 114 described with respect to FIG. 1.

Referring to a first representative embodiment showing the relationship between the balloon catheter and the aspiration catheter in FIG. 10, aspiration catheter 352 and balloon catheter 356 are shown schematically in cross section with no attachment between the two structures, although catheters 352, 356 can be held adjacent each other with a band, clip or other physical attachment. Referring to a second representative embodiment in FIG. 11, aspiration catheter 352 and balloon catheter 356 are shown with a bond 380 between them, which can be an adhesive bond, heat bond or the like.

Referring to FIG. 12, an embodiment is shown in which the balloon can be releasably or fixedly attached with the aspiration catheter. Specifically, balloon 384 is mounted on a tube 386 and is connected to balloon catheter 388. Aspiration catheter 390 had connectors 392 that correspond with fasteners 394 attached to balloon 384 and/or tube 386. Tube 386 is sized to fix over aspiration catheter 390. Balloon 384 can be positioned over aspiration catheter 390 such that connectors 392 engage fasteners 394 to hold balloon 384 in place on aspiration catheter 390. Thus, aspiration catheter 390 can be designed for use with or without a balloon. If the system is disposable, the engagement of connectors 392 with fasteners 394 may not be easily releasable. Suitable connectors 392 and fasteners 394 may include, for example, conventional elements such as bolts and nuts, locking clamps and the like. Thus, in this embodiment, the catheter can be used with or without the balloon, and if used with a balloon, the size of the balloon can be selected a short time prior to use.

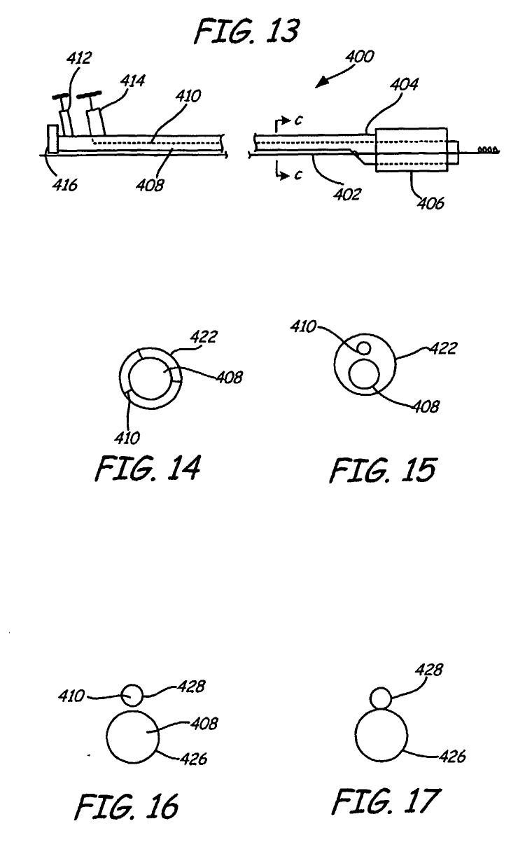

Referring to FIG. 13, an embodiment of a combined medical device having a rapid exchange configuration is depicted. Combined medical device 400 comprises a guide structure 402 and an aspiration catheter 404. Suitable guide structures are described above. Aspiration catheter 404 comprises a rapid exchange section 406, aspiration lumen 408, balloon lumen 410, aspiration connection 412, balloon actuator 414 and distal portion 416. In general, aspiration connection 412, balloon actuator 414 and distal portion 416 can be comparable structures to the corresponding structures in FIGS. 1 and 9.

Rapid exchange section 406 generally comprises a port 420 through which guide structure 402 can pass. Port 420 can be sized such that the diameter of the corresponding guide structure 402 fills the majority of the diameter of the port to enable suction to occur at the distal tip. Also, to direct guide structure 402 to the side port 420 during loading, a small thin walled loading tube may be used. This tube tracks the wire into the major lumen and out the port. After loading, the tube can be removed and generally discarded. In alternative embodiments, multiple lumens can be used to facilitate loading and to isolate the side port from suction.

Aspiration lumen 408 and balloon lumen 410 can be within a single catheter element 422 as shown in two different configurations in FIGS. 14 and 15. In alternative embodiments, aspiration lumen 408 and balloon lumen 410 can be respectively located in an aspiration catheter 426 and balloon catheter 428, as shown in FIG. 16. Aspiration catheter 426 and balloon catheter 428 can be associated together as shown in FIG. 17.

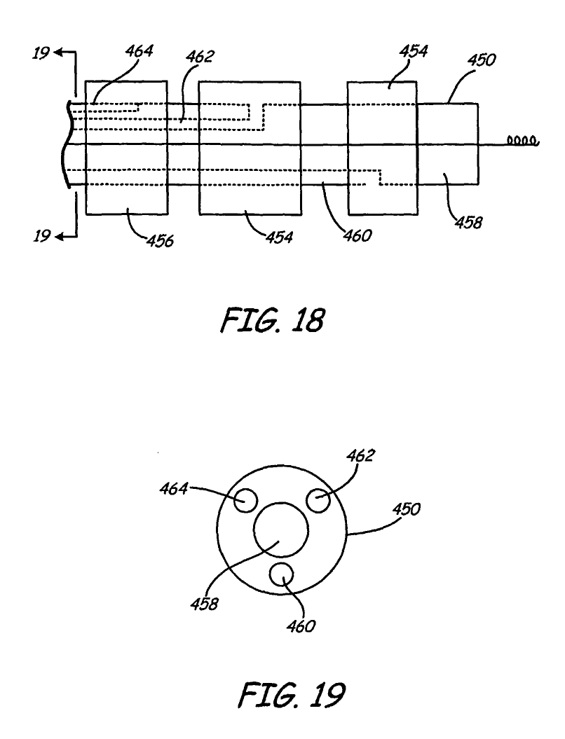

As noted above, the combined medical devices can comprise a plurality of extendable elements. Referring to FIG. 18, aspiration catheter 450 is associated with extendable elements 452, 454, 456. Aspiration catheter 450 can have an aspiration lumen 458, and balloon lumen 460, 462, 464 associated respectively with treatment structures 452, 454, 456. A sectional view is shown in FIG. 19 for one embodiment of the balloon lumen within the aspiration catheter. Extendable elements 452, 454, 456 can be balloons, stents, combinations thereof, or other extendable elements. Balloon lumen 460, 462, 464 can be associated at the proximal end with appropriate actuator elements. While shown in FIG. 18 with the balloon lumen within the aspiration lumen, corresponding structures with the balloon lumen separated from the aspiration catheter, similar to the structures shown in FIG. 9. Furthermore, the aspiration catheter associated with a plurality of medical devices can be an over the wire or rapid exchange configuration. While the device in FIG. 18 is shown with three extendable elements, devices with two extendable elements, four extendable elements or more extendable elements can be designed based on the disclosure herein.

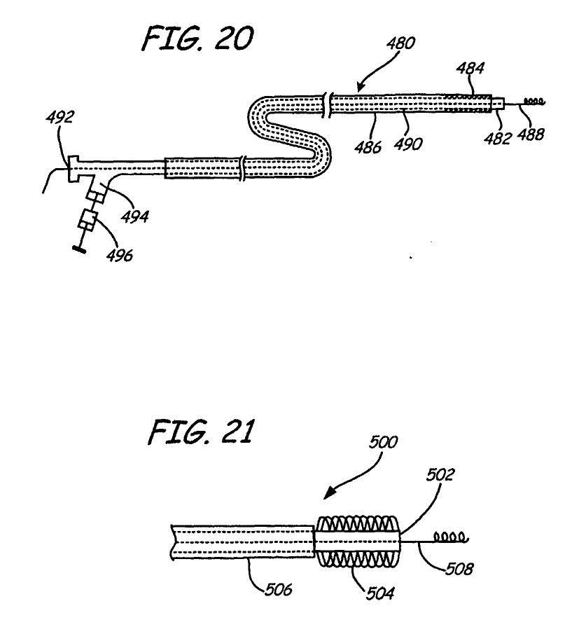

An embodiment based on an extendable stent without a balloon is shown in FIG. 20. Combined medical device 480 comprises an aspiration catheter 482, a self-extending stent 484, a sheath 486 and a guiding structure 488. Aspiration catheter has an aspiration lumen 490, a proximal end 492 and aspiration connection 494 for interfacing with suction device 496. Self-extendable stent 484 is designed to convert to an extended configuration upon withdrawal of sheath 486. An appropriate self-extending stent is described, for example, in U.S. Pat. No. 6,245,103 to Stinson, entitled “Bioabsorbable Self-Expanding Stent,” incorporated herein by reference. Sheath 486 can be tubular, although the inner lumen does not need to be isolated from its exterior since the lumen of sheath 486 is not necessarily used for any purpose. Similarly, sheath 486 can have a non-tubular construction as long as the sheath appropriately restrains stent 484 before deploying the stent and the sheath rides over the aspiration catheter. Suitable guiding structures are described above, and an embolism protection device can be similarly used with the device.

An alternative embodiment of a device with a self-extending stent is shown in FIG. 21. Combined medical device 500 comprises aspiration catheter 502, extendable stent 504, sheath 506 and guiding structure 508. In this embodiment, self-extending stent 504 converts to an extended configuration if the ends are drawn together. A suitable stent is described further in U.S. Pat. No. 5,628,788 to Pinchuk, entitled “Self-Expanding Endoluminal Stent-Graft,” incorporated herein by reference. In this embodiment, to deploy the stent, sheath 506 is directed in a proximal-to-distal direction to push against stent 504.

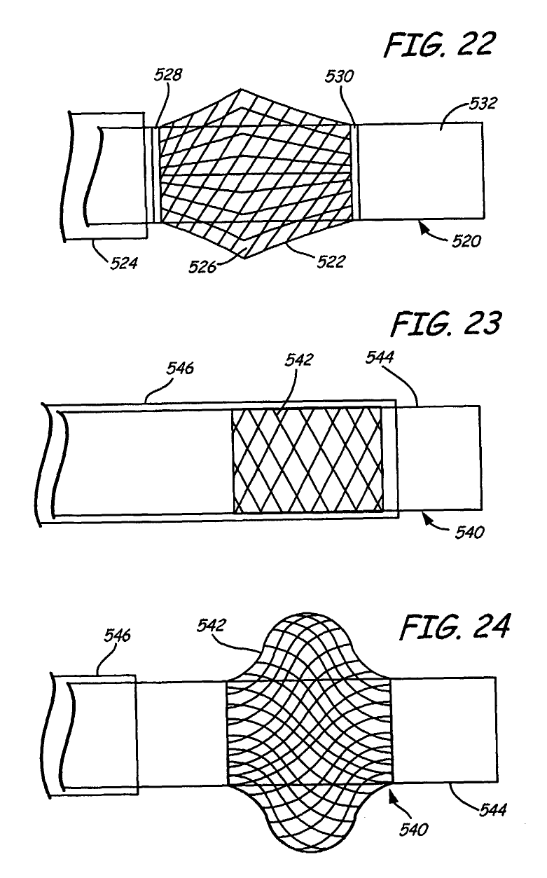

The balloons described above can be used as flow modifiers when their volumes upon expansion are selected to not occlude the vessel completely when deployed. Also, self-extending structures can also be used as flow modifiers. A first embodiment is shown in FIG. 22. In this embodiment, aspiration catheter 520 comprises self-extending flow modifier 522. Self-extending flow modifier 522 can be deployed optionally with a sheath 524. Sheath 524 can similarly be used to depress flow modifier into a lower profile configuration during recovery of catheter 520. Self-extending flow modifier 522 can comprise a spring metal, a shape memory polymer or other similar material than can be compressed while recovering its shape when released. The material of flow modifier 522 can be constructed to have an inherently restrictive structure, and/or the self-extending structure can be associated with a membrane 526 or the like that deflects flow from the surface of the structure. Connecting portions 528, 530 of flow modifier 522 attach flow modifier 522 to catheter tube 532. In alternative embodiments, a flow modifier extending around the circumference of the aspiration catheter may only be attached at one band or other attachment configurations may be suitable for different configurations of a particular flow modifier. Connecting portions 528, 530 can comprise mechanical fasteners, such as bands or clamps, adhesive, combinations thereof, or the like. The extent of the flow modifier along the catheter surface and the outward extent from the catheter surface when deployed can be selected to provide a desired degree of decrease in the flow rate past the flow modifier.

A similar embodiment is shown in FIGS. 23 and 24. Referring to FIG. 23, aspiration catheter 540 comprises an expansive flow modifier 542 on aspiration tube 544. Flow modifier 542 is held in a low profile configuration with sheath 546. In this embodiment, expansive flow modifier 542 comprises a suitable biocompatible metal formed in a shape set braid or a laser cut hypotube. The structure can optionally further comprise an elastomeric covers, which can be formed, for example, from a silicone polymer or polyurethane, to deflect flow from the surface of the structure. Referring to FIG. 24, upon release from the sheath, extendable flow modifier 542 converts into an extended configuration in which flow is modified due to extension of flow modifier 542 partially across the flow.

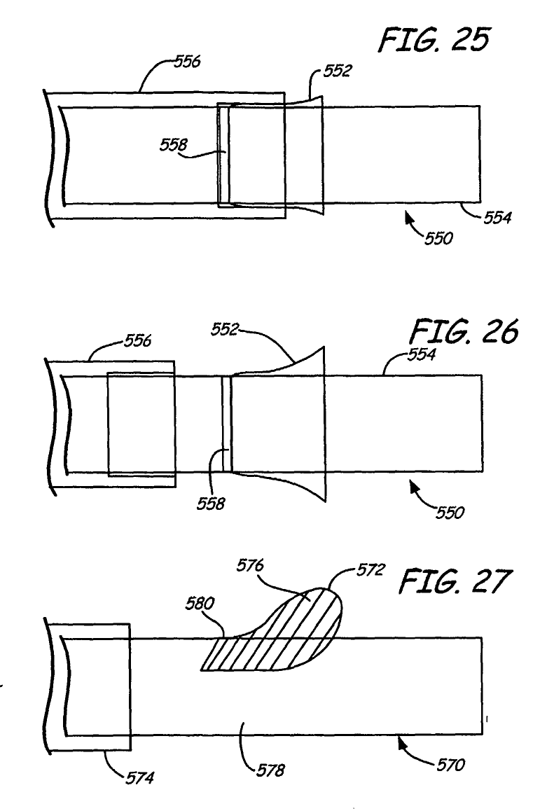

Another self-extending embodiment is depicted in FIGS. 25 and 26. Referring to FIG. 25, aspiration catheter 550 comprises a flaring flow modifier 552 on aspiration tube 554. Sheath 556 constrains flow modifier 552 into a low profile configuration for deployment and/or retrieval through riding over at least the proximal end of flow modifier 552, as shown in FIG. 25. Flow modifier 552 is attached to tube 554 at its proximal end at fastener 558. Fastener 558 can be a mechanical fastener, such as a band or a clamp, and/or an adhesive fastener or the like. Referring to FIG. 26, if sheath 556 is moved in a proximal direction relative to tube 554, flow modifier flairs outward at its distal side since fastener 558 constrains flow modifier 552 at its proximal side.

Flow modifiers 522, 542, 552 in FIGS. 22, 24 and 26, respectively, extend around the circumference of aspiration catheter tubes, although each flow modifier may or may not have cylindrical symmetry. However, in alternative embodiments, a self-extending flow modifier may not extend around the circumference of the aspiration catheter. Referring to FIG. 27, aspiration catheter 570 comprises a self-extending flow modifier 572 that does not extend around the circumference of aspiration catheter 570. An optional sheath 574 can be used to deploy flow modifier 572 by moving sheath 574 in a proximal direction relative to aspiration catheter 570 to release flow modifier 572. Self-extending flow modifier 572 generally comprises a resilient material, such as a spring metal or shape memory polymer, and may comprise an optional membrane 576 or the like to contribute to restricting flow past the flow modifier. As shown in FIG. 27, flow modifier 572 has a flap shape attached to catheter tube 578 along attachment portion 580, which can comprise a mechanical fastener, such as a band or clamp and/or an adhesive or the like. In other embodiments, flow modifiers that do not extend around the circumference can have different shapes than the flap structure shown in FIG. 27, and such flow modifiers may be attached to the catheter tube along two attachment portions or other suitable attachment configuration based on the design of the flow modifier. Again, the size and shape of the flow modifier can be selected to achieve the desired degree of flow modification.

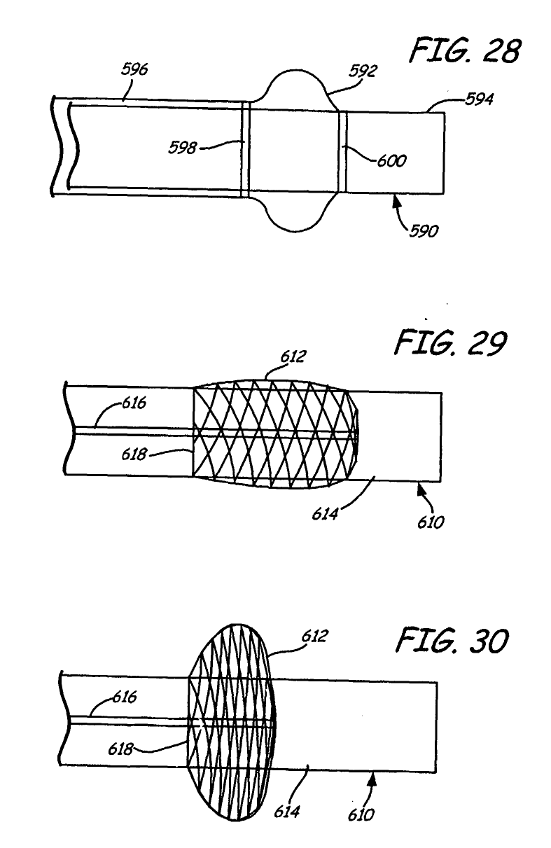

Additional non-self-extending structures are suitable for forming and deploying flow modifiers. A first representative embodiment is shown in FIG. 28 in a deployed configuration. Aspiration catheter 590 comprises flow modifier 592 on aspiration tube 594. Sheath 596 is configured to interface with proximal edge 598 of flow modifier 592. Proximal edge 598 is configured to slide along tube 594. Fastener 600 holds the distal end of flow modifier 592 at a fixed position along tube 594. Fastener 600 can comprise a mechanical fastener, an adhesive fastener or the like or combinations thereof. For deployment, flow modifier 594 generally is in a low profile configuration. Movement of sheath 596 in a distal direction relative to tube 594 results in proximal edge 598 being moved toward fastener 600 such that the center of flow modifier 592 flairs outward into a deployed configuration, as shown in FIG. 28. In some embodiments, flow modifier 592 is resilient such that flow modifier resumes a low profile configuration for removal from the patient if sheath 596 no longer applies force against proximal edge 598.

A further embodiment of an actuatable flow modifier is shown in FIGS. 29 and 30. Aspiration catheter 610 comprises flow modifier 612 over tube 614. Flow modifier 612 is operably connected to actuation wire 616. Flow modifier 612 is attached to tube 614 at proximal end 618. Actuation wire 616 can function as a pull wire, push wire or both. In some embodiments, actuation wire 616 is formed from a suitable biocompatible metal. For example, flow modifier 612 can comprise a resilient material with a natural shape in a low profile configuration shown in FIG. 29 such that flow modifier 612 is deployed using wire 616 as a pull wire that can be used to deploy flow modifier as shown in FIG. 30. Alternatively, flow modifier 612 can have a natural shape in an extended configuration as shown in FIG. 30 such that wire 616 functions as a push wire to stretch flow modifier 612 into a low profile configuration of FIG. 29 for delivery and/or recovery. In further embodiments, flow modifier 612 can comprise a malleable but non-resilient material such that actuation wire 616 can be used to transform flow modifier between the configurations shown in FIGS. 29 and 30 without flow modifier 612 having a particular natural configuration. In alternative embodiments, the configuration of flow modifier 612 can be reversed such that it has a fixed distal end and an actuation wire moves the proximal side of the flow modifier to form a desired configuration. Also, actuation wires can be used to deploy flow modifiers that do not extend around the circumference of the aspiration tube. For example, the embodiment in FIG. 27 can be adapted for control with an actuation wire rather than the sheath.

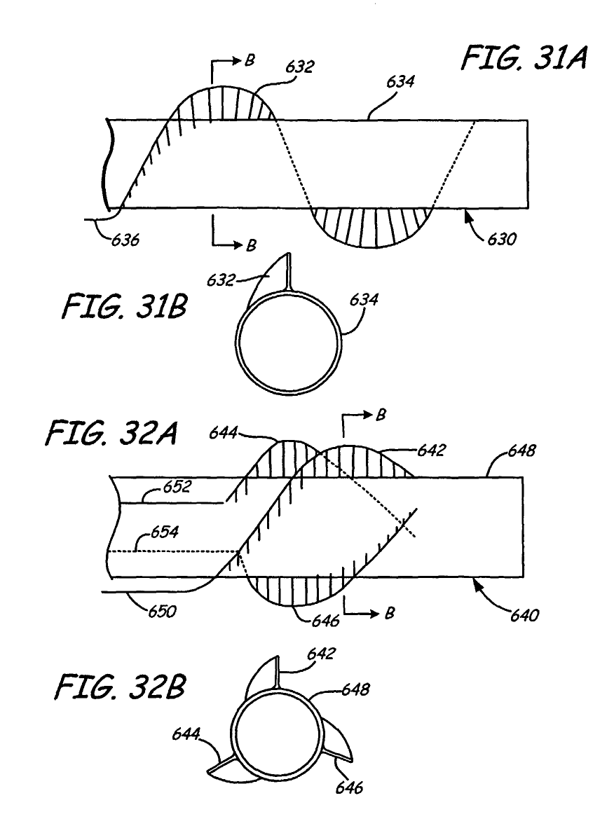

Spiral shaped flow modifiers are shown in two embodiments in FIGS. 31 and 32. Referring to FIGS. 31A and 31B, aspiration catheter 630 comprises a spiral fin 632 over aspiration tube 634. Upon deployment, spiral 632 disrupts and slows flow in a vessel past the spiral fin. Spiral fin 632 can be resilient such that a pull wire 636 can be used to collapse the spiral fin during deployment and/or retrieval. Release of pressure on pull wire 636 then releases spiral fin 632 to resume its natural extended position. In alternative embodiments, a sheath can be used to collapse spiral fin 632 into a low profile configuration.

Referring to FIGS. 32A and 32B, aspiration catheter 640 comprises three spiral diverters 642, 644, 646 symmetrically distributed around the circumference of aspiration tube 648, although asymmetrical distributions can be used if desired. Spiral diverters 642, 644, 646 divert and slow flow past them within a vessel. Pull wires 650, 652, 654 can be used, respectively, to deflect resilient diverters into a lower profile configuration for delivery and/or retrieval of the device. When tension is released on pull wires 650, 652, 654, diverters 642, 644, 646 are released to resume their extended configurations. In alternative embodiments, a sheath can be used to collapse diverters 642, 644, 646 into lower profile configurations. While FIGS. 31 and 32 depict one or three spiral diverters, two, four or more can be used if desired. Similarly, while the diverters in FIGS. 31 and 32 are spiral, other shapes can also be used to modify and slow the flow past the aspiration aperture.

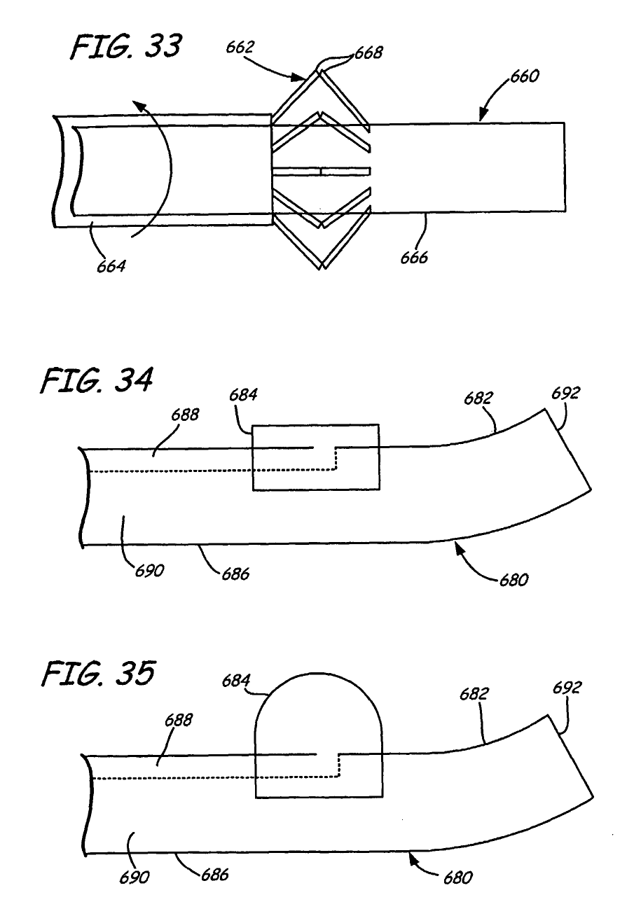

FIG. 33 depicts an aspiration catheter 660 comprising a flow modifier 662 operably connected to a sheath 664 that rotates round tube 666 of aspiration catheter 660. Flow modifier 662 comprises a plurality of interconnected struts 668 that are configured to deform collectively through rotation between a low profile configuration and an extended configuration that projects outward form tube 666. Thus, rotation of sheath 664 relative to tube 666 can be used to deploy or contract flow modifier 662. Struts 668 may or may not be associated with a membrane to restrict flow between the struts.

FIGS. 34 and 35 depict an aspiration catheter with an inflatable flow modifier that does not extend around the circumference of the catheter. Referring to FIG. 34, aspiration catheter 680 comprises a curved tip 682 and a balloon 684 mounted over a portion of the circumference of aspiration tube 686. Balloon 684 is in fluid communication with balloon lumen 688. Balloon lumen 688 extends to a fluid source at the proximal end not shown, but which can be similar to the proximal end shown in FIG. 1, for example. Aspiration lumen 690 extends from a suction device at the proximal end to aspiration opening 692. As shown in FIG. 34, balloon 684 is deflated. Referring to FIG. 35, balloon 684 is inflated into a configuration that extends the balloon in a direction toward the curve of the tip 682. Thus, balloon 684 diverts flow away from aspiration opening 692 such that aspiration is more effective to remove debris from upstream from the aspiration opening. In other embodiments, it may be desirable to have the tip of the catheter bent the other direction relative to a flow modifier to direct the reduced flow in the vessel toward the aspiration opening. The use of curved aspiration catheter tips to improve tracking of aspiration catheters along a guide structure is described further in copending U.S. patent application Ser. No. 11/207,169 to Boldenow et al., entitled “Improved Tracking Aspiration Catheter,” incorporated herein by reference.

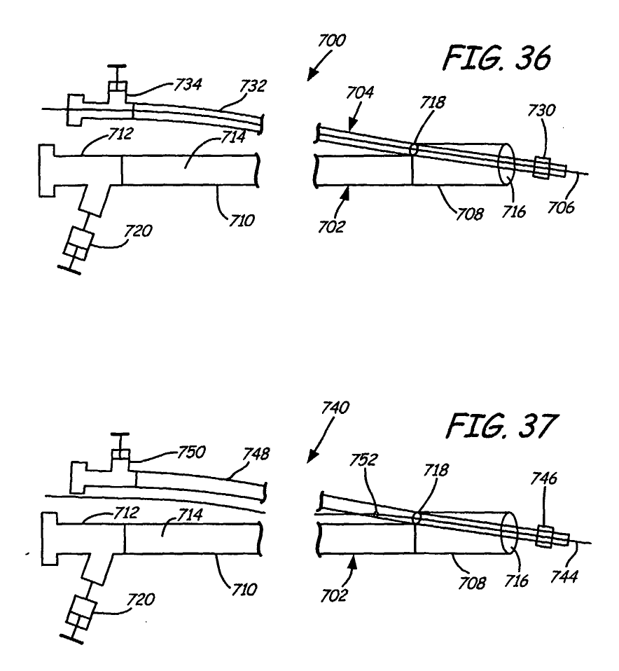

FIGS. 36 and 37 depict another approach to the combination of a treatment structure with an aspiration catheter. In the approaches of these figures, there is more flexibility in the design options. For example, a conventional treatment structure can be combined with an appropriately designed aspiration catheter in some embodiments.

Referring to FIG. 36, medical device 700 comprises aspiration catheter 702, treatment catheter 704 and guide structure 706. Aspiration catheter 702 comprises a rapid exchange segment 708 connected to shaft 710 and proximal segment 712 to form an aspiration lumen 714. Rapid exchange segment comprises an aspiration opening 716 at its distal end and a rapid exchange port 718 adjacent the point of attachment to shaft 710. Proximal segment 712 comprises a suction device 720 that can connect to aspiration lumen 714. Suction device can be a syringe or other suitable suction device, which may be replaceable with a Leur lock or other attachment device. Treatment catheter 704 comprises treatment structure 730, shaft 732 and actuation element 734. Treatment structure 730 can comprise a balloon, a stent or a combination thereof. In some embodiments, a balloon catheter extends from actuation element 734 to treatment structure 730, and then actuation element can comprise a syringe or other structure to deliver an expansion fluid to a balloon or the like to a balloon. In alternative embodiments, the treatment structure can be a self-extending stent or the like and the actuation element 734 can be a suitable structure, such as a sheath, a pull wire or the like. Rapid exchange port 718 generally is sized slightly larger than shaft 732 such that there is no significant gap when the devices are deployed that would interfere with applying suction from aspiration opening 716. In this embodiment, treatment catheter 704 runs over guide structure 706.

Referring to FIG. 37, medical device 740 comprises an equivalent aspiration catheter 702 as shown in FIG. 36 along with treatment catheter 742 and guide structure 744. Treatment catheter 742 comprises treatment structure 746, shaft 748 and actuation element 750. Treatment structure 742 and actuation element 750 can be equivalent to treatment structure 730 and actuation element 734 of FIG. 36. Referring to FIG. 37, shaft 748 comprises a rapid exchange port 752 through which guide structure 744 can exit shaft 748. A balloon lumen or other component attached to the actuation element 750 can extend from actuation element 734 to treatment structure 744. This embodiment of the combined medical device combines the advantages of a rapid exchange configuration with the ability to use a separate treatment structure, such as a conventional balloon or stent delivery system with an aspiration catheter.

Materials for Devices

With respect to the components of the medical device that contact a patient's bodily fluids, suitable materials are generally biocompatible in that they are non-toxic, non-carcinogenic and blood compatible and do not induce hemolysis or a significant immunological response. In addition, the materials may have significant desired properties to balance mechanical strength with flexibility. Furthermore, the treatment devices can be associated with therapeutic compounds that are selected for local delivery.

The aspiration catheters, sheaths and balloon catheters can be formed from one or more biocompatible materials, including, for example, metals, such as stainless steel or alloys, e.g., Nitinol®, or polymers such as polyether-amide block co-polymer (PEBAX®), nylon (polyamides), polyolefins, polytetrafluoroethylene, polyesters, polyurethanes, polycarbonates or other suitable biocompatible polymers. Radio-opacity can be achieved with the addition of markers, such as platinum-iridium or platinum-tungsten or through radio-pacifiers, such as barium sulfate, bismuth trioxide, bismuth subcarbonate, powdered tungsten, powdered tantalum or the like, added to the polymer resin. Generally, different sections of an aspiration catheter or balloon catheter can be formed from different materials from other sections, and sections of a catheter can comprise a plurality of materials at different locations and/or at a particular location. In particular, it may be desirable to form distal compartment of an aspiration catheter or a portion thereof from an elastomeric polymer, such as suitable polyurethanes, polydimethyl siloxane and polytetrafluoroethylene. In addition, selected sections of the catheter can be formed with materials to introduce desired stiffness/flexibility for the particular section of the catheter. Similarly, fittings can be formed form a suitable material, such as one or more metals and/or one or more polymers.

In general, the components of an integrated guiding structure or other guiding structure, such as a guidewire, can be formed from one or more of various material, such as polymers, metals and combinations thereof. A tube and corewire of an integrated guiding structure may or may not be formed from the same material. Suitable biocompatible metals include, for example, titanium, cobalt, stainless steel, nickel, iron alloys, cobalt alloys, such as Elgiloy®, a cobalt-chromium-nickel alloy, MP35N, a nickel-cobalt-chromium-molybdenum alloy, and Nitinol®, a nickel-titanium alloy.

Suitable polymers include, for example, synthetic polymers as well as purified biological polymers and combinations thereof. Suitable synthetic polymers include, for example, polyamides (e.g., nylon), polyesters (e.g., polyethylene teraphthalate), polyacetals/polyketals, polystyrenes, polyacrylates, vinyl polymers (e.g., polyethylene, polytetrafluoroethylene, polypropylene and polyvinyl chloride), polycarbonates, polyurethanes, poly dimethyl siloxanes, cellulose acetates, polymethyl methacrylates, polyether ether ketones, ethylene vinyl acetates, polysulfones, nitrocelluloses, similar copolymers and mixtures thereof. Based on desirable properties and experience in the medical device field, suitable synthetic polymers include, in particular, polyether ether ketones, polyacetals, polyamides (e.g., nylons), polyurethanes, polytetrafluoroethylene, polyester teraphthalate, polycarbonates, polysulfone and copolymers and mixtures thereof.

In other embodiments, the surface of the corewire, the inner surface of the tube, the outer surface of the tube or portions thereof is coated with a friction reducing agent. Suitable friction reducing agents include, for example, suitable polymers, such as polytetrafluorethylene, i.e., Teflon® or parylene. The coating of the corewire can facilitate relative longitudinal motion of the corewire relative to the tube.

Balloons can be formed from elastic or non-elastic materials. Generally, the balloon is formed from polymers. Suitable polymers include, for example, nylon (polyamides), polyethylene, other polyolefins, polyethylene terephthalate, polyvinylchloride, polyarylene sulfide, silicone rubber, latex rubber, Arnitel® copolyester elastomers, Hytrel® polyester elastomer, polyetherether ketone, Pebax® polyether block amides, Teflon®, other thermoplastic elastomers or combinations thereof. These can be formed using conventional polymer processing approaches, such as extrusion, blow molding, compression molding, calendering and the like.

Appropriate medical adhesives should be biocompatible, in that they are non-toxic, non-carcinogenic and do not induce hemolysis or an immunological response. In general, the adhesive can be a single component adhesive or multi-component adhesive. Further suitable adhesives include synthetic adhesives, natural adhesives and combinations thereof. For example, suitable biocompatible adhesives include commercially available surgical adhesives, such as one component cyanoacrylate adhesives (such as 2-octyl cyanoacrylate, Dermabond™, from Ethicon Products), fibrin glue (such as Tissucol® from Baxter) and mixtures thereof. Suitable two-component synthetic adhesives include, for example, urethane-based polymers, copolymers, and mixtures thereof. Polyurethanes are ester-amide derivatives of carboxylic acids.

Stents can be formed from stainless steel, tantalum, shape memory alloys, polymers and coated versions thereof. Stents may be balloon extendable or self-extendable. Balloon extendable stents can be crimped to the balloon for delivery. Some balloon-stent structures are described further, for example, in U.S. Pat. No. 6,106,530, entitled “Stent Delivery Device,” U.S. Pat. No. 6,364,894, entitled Method of Making an Angioplasty Balloon Catheter,” and U.S. Pat. No. 6,156,005, entitled “Ballon [sic] Catheter For Stent Implantation,” each of which are incorporated herein by reference.

Drug coated stents are presently commercially available, such as the pacitaxel eluting Taxus™ stent from Boston Scientific and the sirolimus eluting Cypher™ stent from Johnson & Johnson. Stents and balloons associated with therapeutic agents are described further in U.S. Pat. No. 6,491,617 to Ogle et al., entitled “Medical Articles That Resist Restenosis,” incorporated herein by reference.

The materials can be processed, for example, using conventional techniques, such as extrusion, molding, machining, calendering, and combinations thereof. In general, a combination of the materials can be combined in various ways into the devices described above to achieve the properties of the resulting device.

Use of Combined Device

The combined devices described herein are designed for association of an aspiration catheter with one or more extendable devices. The extendable devices can be a treatment structure and/or a flow modifier. For embodiments based on a treatment structure, the application of suction can be performed in association with the deployment of a balloon, delivery of a stent or a combination thereof. The medical device can be designed such that the components are appropriately positioned for a particular procedure. For embodiments involving a flow modifier, generally the flow modifier is in a deployed configuration when suction is applied through the aspiration catheter. Suitable embodiments of the combined medical device may or may not be further associated with an embolism protection device.

Emboli can be generated as a result of the delivery of an angioplasty balloon, a stent or other intervention to open a constriction in a vessel within a patient. One approach to prevent some or all of these emboli from flowing past the intervention location in a vessel and blocking smaller vessels involves that application of aspiration to suck the emboli within an aspiration catheter and remove the emboli from the body. In some embodiments, aspiration is initiated at or shortly before the deployment of a treatment structure. However, delivery of the aspiration catheter and treatment structure can itself disrupt material blocking the vessel. In these embodiments, it may be desirable to start the aspiration while the catheter and/or treatment structure is inserted past the blockage. The suction can be applied intermittently as appropriate.

A flow modifier is selected to improve the effectiveness of aspiration with respect to the removal of emboli. In some embodiments, to have a comfort level that emboli within the vessel are being captured the aspiration rate with respect to volume aspirated per unit time should be at least as high as minimum flow rates through the vessel over the pulse cycle within the vessel. In some embodiments, the degree of extension of the flow modifier can be selected based on the size of the vessel. For example, the amount of volume expansion of a balloon can be selected to reduce flow after expansion a desired amount for the vessel. Similarly, a flap attached to an actuation element may be similarly extended a selected amount based on the degree of movement of the actuation element. Thus, the nature of the aspiration within the vessel can be controlled to balance various factors.

In some embodiments, the combined medical device further comprises an embolism protection device. The embolism protection device can be an occlusive or partially occlusive device. Suit occlusive devices can comprise, for example, a balloon. In some embodiments, occlusive devices can be used on both sides of the treatment device to isolate the portion of the vessel with the treatment device, such that the isolated region can be aspirated for the removal of emboli. The use of occlusive devices to isolate a treatment structure is described further in U.S. Pat. No. 6,454,741 to Muni et al., entitled “Aspiration Method,” incorporated herein by reference.

In further embodiments of particular interest, the combined medical device comprises a filter-based embolism protection device. Specific filters based on surface capillary fibers are described in detail above. Animal studies have demonstrated excellent filtering properties for fiber-based filters with surface capillary fibers, as described above. Other filter-based embolism protection devices are commercially available. The fiber-based devices described above are particularly suitable due to their incorporation into an integrated guide device. The filter structure can be positioned and opened/deployed prior to placement of the treatment structure at the restricted flow region. If the filter is opened prior to placement of the treatment device at the restricted flow location, then any debris that is generated during placement of the treatment structure can be collected by the filter. Aspiration may or may not be used during the delivery and/or deployment of the treatment structure.

In some embodiments, aspiration is applied during the recovery of the filter, although aspiration may or may not be applied during earlier portions of the procedure. If a surface capillary fiber-based filter is deployed, generally all emboli of significance are collected while the filter is deployed such that aspiration prior to recovery of the filter may not be used. When the filter is converted into configuration for removal from the vessel, emboli may be released from the filter. For the fiber-based filters described above, the filters can be converted to a recovery configuration by moving the corewire distal relative to the tube over the corewire. The application of aspiration during the recovery of the filter can reduce or eliminate the release of any emboli.

Aspiration can be started shortly before or at the time of initiating the recovery of the filter. The fiber-based filter described above can be withdrawn into a distal compartment of the aspiration catheter to facilitate recovery of the filter. For these embodiments, aspiration can be continued until the filter is withdrawn partly or fully within the distal compartment. The use of a flow modifier can help to capture all or a greater portion of any emboli released during recovery of the filter. Uses of an aspiration catheter to recover a fiber-based embolism protection devices are described further in copending U.S. patent application Ser. No. 10/854,920 to Galdonik et al., entitled “Emboli Filter Export System,” incorporated herein by reference.

Distribution and Packaging

The medical devices described herein are generally packaged in sterile containers for distribution to medical professionals for use. The articles can be sterilized using various approaches, such as electron beam irradiation, gamma irradiation, ultraviolet irradiation, chemical sterilization, and/or the use of sterile manufacturing and packaging procedures. The articles can be labeled, for example with an appropriate date through which the article is expected to remain in fully functional condition. The components can be packaged individually or together.

Various devices described herein can be packaged together in a kit for convenience. For example, an aspiration catheter can be packaged along with an integrated system for delivery and recovery of an embolism protection device. The kit can further include, for example, labeling with instruction for use and/or warnings, such as information specified for inclusion by the Food and Drug administration. Such labeling can be on the outside of the package and/or on separate paper within the package.

The embodiments described above are intended to be illustrative and not limiting. Additional embodiments are within the claims. Although the present invention has been described with reference to preferred embodiments, workers skilled in the art will recognize that changes may be made in form and detail without departing from the spirit and scope of the invention.

1. A method for retrieving an embolism protection device having a low profile configuration and an extended deployed configuration, the method comprising withdrawing the embolism protection device into an aspiration catheter within a patient's vessel while aspiration is being applied, wherein an extendable device associated with the aspiration catheter is extended and partially occludes flow relative to the flow in which the extendable device does not have an extended configuration, wherein the extendable device lacks cylindrical symmetry around the aspiration catheter.

2. The method of claim 1 wherein the embolism protection device comprises a bundle of fibers which are stretched in the low profile configuration and bent in the deployed configuration.

3. The method of claim 1 wherein the aspiration catheter has a bent tip.

4. The method of claim 1 wherein the extendable device comprises a balloon.

5. The method of claim 1 wherein the balloon extends around the entire circumference of the aspiration catheter.

6. The method of claim 1 wherein the balloon extends only partially around the circumference of the aspiration catheter.

7. The method of claim 1 wherein the extendable device comprises a flap.

8. An aspiration catheter comprising a suction connection, a proximal portion operably connected to the suction connection, a shaft and a self-extending structure, the shaft being operably connected to the proximal portion and having an aspiration opening connected to an aspiration lumen in fluid communication with the suction connection wherein the self-extending structure is within about 20 centimeters of the aspiration opening.

9. The aspiration catheter of claim 8 further comprising a sheath that removably covers the self-extending structure.

10. The aspiration catheter of claim 8 wherein the self-extending structure comprises a self-extending stent.

11. The aspiration catheter of claim 8 wherein the self-extending structure comprises a shape memory structure connected to an outer surface of the shaft to extend into a partially occlusive structure.

12. The aspiration catheter of claim 11 wherein the shape memory structure comprises a shape memory metal.

13. The aspiration catheter of claim 11 wherein the shape memory structure comprises a shape memory polymer.

14. The aspiration catheter of claim 11 wherein the self-extending structure extends around the entire outer circumference of the aspiration catheter.

15. The aspiration catheter of claim 11 wherein the self-extending structure extends around only a portion of the aspiration catheter's out circumference.

16. The aspiration catheter of claim 11 wherein the self-extending structure further comprises a membrane supported by the shape memory structure.

17-33. (canceled)

34. A medical device for performing less invasive medical procedures within a patient, the device comprising an aspiration catheter, a balloon apparatus comprising a balloon element and a balloon catheter, the aspiration catheter having an aspiration lumen operably connected to a suction device and an aspiration opening at the distal end of the aspiration lumen, wherein the aspiration opening is distal to the balloon element and wherein the aspiration opening has an area greater than the average cross section of the aspiration lumen.

35. The medical device of claim 34 wherein the balloon element comprises cylindrically symmetric flexible element.

36. The medical device of claim 34 further comprising a stent associated with the balloon element.

37. The medical device of claim 34 wherein the catheter element is operably connected to a fluid source that is configured to deliver fluid pressure to the balloon.

38. The medical device of claim 34 wherein the aspiration catheter comprises a suction device, a proximal portion, a rapid exchange segment having a distal end and a shaft attached between the proximal portion and the rapid exchange segment, wherein the rapid exchange segment comprises a port adjacent the connection between the shaft and the rapid exchange segment.

39. The medical device of claim 38 further comprising a guide structure extending through the port.

40. The medical device of claim 38 further comprising a guide structure and a filter structure connected to the guide device.

41. The medical device of claim 40 wherein the filter structure comprises surface capillary fibers.

42. The medical device of claim 40 wherein the guide structure comprises a guide wire and a tube extending over the guide wire wherein a torque coupler connects the guide wire and the tube.

43. The medical device of claim 40 wherein the aspiration catheter has an extended compartment at its distal end into which the filter can be brought for removal.

44. The medical device of claim 40 wherein the catheter element is connected to the aspiration catheter.

45. The medical device of claim 40 wherein the catheter element is disconnected from the aspiration catheter.

46. The medical device of claim 38 wherein the aspiration opening is at the distal end of a distal compartment that has an average diameter greater than the average diameter of the shaft and wherein the balloon is mounted on the distal compartment.

47. A medical device for performing less invasive medical procedures within a patient, the device comprising a guide structure, an aspiration catheter, a balloon element mounted on the aspiration catheter and a fluid source operably connected to the balloon element, wherein the aspiration catheter comprises a suction device, a proximal portion, a rapid exchange segment having a distal end and a shaft attached between the proximal portion and the rapid exchange segment, wherein the rapid exchange segment comprises a port adjacent the connection between the shaft and the rapid exchange segment, wherein the guide structure passes through the port and wherein the rapid exchange segment has a single lumen.

48. The medical device of claim 47 wherein the rapid exchange segment has a length of at least about 10 centimeters.

49. The medical device of claim 47 wherein the suction device is operative coupled to the proximal portion and a continuous lumen extends from the proximal portion through the shaft to the distal end.

50. The medical device of claim 47 wherein the suction device comprises a syringe.

51. The medical device of claim 47 wherein the suction device comprises a pump.

52. The medical device of claim 47 wherein the shaft has an approximately constant diameter.

53. The medical device of claim 47 wherein the shaft has a diameter from about 0.020 inches to about 0.035 inches.

54. The medical device of claim 47 wherein the shaft has a length of at least about 100 cm.

55. The medical device of claim 47 wherein the rapid exchange segment comprises a distal tip with an extended compartment at the distal end of the rapid exchange segment.

56. The medical device of claim 47 further comprising a balloon catheter connecting the fluid source and the balloon element.

57. The medical device of claim 47 wherein the aspiration catheter comprises a balloon lumen fluidly connecting the balloon element and a balloon actuator.

58-67. (canceled)

68. A medical device comprising an aspiration catheter, a treatment catheter and a guide structure, wherein the aspiration catheter comprises a rapid exchange segment, a proximal section, a shaft connected between the rapid exchange segment and the proximal section to form an aspiration lumen extending between the proximal section and the rapid exchange section, and a suction device connected to the proximal section and in fluid communication with the aspiration lumen, the rapid exchange segment comprising a treatment catheter port, the treatment catheter comprising a shaft, a treatment structure mounted on the shaft, and an actuation device operably connected to the treatment structure, wherein the shaft of the treatment catheter extends through the treatment catheter port.

69. The medical device of claim 68 wherein the treatment structure comprises a balloon and the treatment catheter further comprises a balloon lumen operably connected to the balloon.

70. The medical device of claim 68 wherein the treatment structure comprises a stent.

71. The medical device of claim 68 further comprising a guide structure that extends through a majority of the length of the treatment catheter.

72. The medical device of claim 68 further comprising a guide structure and the treatment catheter further comprising a rapid exchange segment extending to the distal end of the treatment catheter and a guide port adjacent the rapid exchange segment along the distal half of the treatment catheter with the guide structure extending through the guide port.

73-75. (canceled)

76. An aspiration catheter comprising a suction connection, a proximal portion operably connected to the suction connection, a shaft and an extendable device, the shaft being operably connected to the proximal portion and having an aspiration opening connected to an aspiration lumen in fluid communication with the suction connection wherein the extendable device lacks cylindrical symmetry around the aspiration catheter.
